(19) United States
(12) Patent Application Publication
(10) Pub. No.: US 2019/0343822 A1
(43) Pub. Date: Nov. 14, 2019

(54) TREATMENT FOR GLAUCOMA

(71) Applicant: INDIANA UNIVERSITY RESEARCH AND TECHNOLOGY CORPORATION, Indianapolis, IN (US)

(72) Inventor: Alex Straiker, Bloomington, IN (US)

(21) Appl. No.: 16/408,817

(22) Filed: May 10, 2019

Related U.S. Application Data
(60) Provisional application No. 62/669,491, filed on May 10, 2018.

(51) Int. Cl. A61K 31/454; A61P 27/06; A61K 31/4439; A61K 9/00

(52) U.S. Cl. CPC A61K 31/454; A61K 9/0048; A61K 31/4439; A61P 27/06

(57) ABSTRACT

Disclosed herein are methods for treating glaucoma and reducing intraocular pressure. Methods for treating glaucoma include administering a cannabinoid receptor antagonist to a subject in need thereof. Methods for reducing intraocular pressure include administering a cannabinoid receptor antagonist to a subject in need thereof. Methods for treating glaucoma also include administering a negative allosteric modulator of a cannabinoid CB1 receptor to a subject in need thereof. Methods for reducing intraocular pressure also include administering a negative allosteric modulator of a cannabinoid CB1 receptor to a subject in need thereof.

### CROSS-REFERENCE TO RELATED APPLICATION(S)

This application claims the benefits of U.S. Provisional Patent Application Ser. No. 62/669491, filed May 10, 2018, the entire contents of which are incorporated herein by reference.

### STATEMENT OF GOVERNMENT SUPPORT

This invention was made with government support under EY024625 awarded by National Institutes of Health (NIH). The Government has certain rights in the invention.

### BACKGROUND

The present disclosure relates generally to treating glaucoma. More particularly, the present disclosure relates to methods for reducing intraocular pressure by long-term administration of CB1 receptor antagonists.

Glaucoma is a disease that threatens an estimated 3 million Americans with permanent blindness. In African Americans, glaucoma is the leading cause of blindness after cataracts. The chief hallmark of glaucoma is elevated intraocular pressure (IOP) and reduction of the IOP remains the chief means of first-line therapeutic intervention. IOP is regulated in part by the endogenous cannabinoid system and activating CB1 receptors lowers ocular pressure.

The current market for glaucoma drugs is −$2 billion/year across the seven largest markets and only one new drug has been approved in the past 20 years. Though there are six classes of drugs available to lower ocular pressure, most of these drugs reduce inflow of aqueous humor, limiting the natural cycling and increasing the risk of cataracts. Moreover because glaucoma requires daily treatments over years, or even decades, side effects and tolerance are serious issues that force older patients to invasive, expensive, and often ineffective surgical procedures (nearly 200,000 each year). Treatments for glaucoma exist, but not for all forms, and often with side-effects. Currently there are six classes of drugs currently approved for therapeutic use in the treatment of glaucoma via IOP reduction, however, there remain incidences of patients who are unresponsive to these medications, or who develop tolerance to existing treatments. Moreover compliance is a key issue for treatment because half of patients stop taking their daily/twice daily drops within the first year. A long-lasting treatment that requires fewer treatments would be beneficial and distinct from all available treatments, particularly long-lasting therapeutics.

Accordingly, there remains a need for additional therapeutics for the treatment of glaucoma and reducing IOP.

### BRIEF DESCRIPTION

The present disclosure is generally related to treating glaucoma. More particularly, the present disclosure relates to methods for treating glaucoma by administration of CB1 receptor antagonist. The present disclosure also relates to methods for reducing intraocular pressure by long-lasting administration of CB1 receptor antagonist.

In one aspect, the present disclosure is directed to a method for treating glaucoma in a subject in need thereof, the method comprising administering a cannabinoid receptor antagonist to the subject.

In one aspect, the present disclosure is directed to a method reducing intraocular pressure in a subject in need thereof, the method comprising administering a cannabinoid CB1 receptor antagonist to the subject.

In one aspect, the present disclosure is directed to a method for treating glaucoma in a subject in need thereof, the method comprising administering a CB1 receptor negative allosteric modulator to the subject.

In one aspect, the present disclosure is directed to a method reducing intraocular pressure in a subject in need thereof, the method comprising administering a CB1 receptor negative allosteric modulator to the subject.

### BRIEF DESCRIPTION OF THE DRAWINGS

The disclosure will be better understood, and features, aspects and advantages other than those set forth above will become apparent when consideration is given to the following detailed description thereof. Such detailed description makes reference to the following drawings, wherein:

FIG. 1A depicts evidence for a second, diurnally expressed, site of action for CB1 in regulation of ocular pressure. FIG. 1A depicts lower baseline ocular pressure in CB1 knockout mice relative to wild type (WT) controls. ***, p<0.001, unpaired t-test.

FIG. 1B depicts the rise in ocular pressure by CB1 antagonist SR141716 ad midday in mice maintained in standard light cycle, but decreases ocular pressure in reverse light cycle. *,p<0.05, p<0.01, paired t-test.

FIG. 1C depicts the rise in ocular pressure by CB1 antagonist SR141716 at midday in mice maintained in standard light cycle (FIG. 1B), but decreases ocular pressure in reverse light cycle. *<p0.05, P<0.01, paired t-test.

FIG. 2A depicts lower pressure in WT mice after treatment with SR141716 as above.

FIG. 2B shows a lack of effect on day 7 after 7 days of daily treatment.

FIG. 2C depicts the baseline ocular pressures at day 1 and 7 in drug and vehicle treated eyes.

FIG. 3A depicts ocular pressure changes in WT mice after topical treatment with negative allosteric modulator PSNCBAM1 showing a non-effect.

FIG. 3B depicts ocular pressure after intraperitoneal injection of PSNCBAM1.

FIG. 4A depicts a single treatment with SR141716 (SR1, 1 mM) does not last 24 hours but does result in a rebound at 48 hrs.

FIG. 4B depicts 7 days daily treatment results in a persistent lowering of IOP (24 hrs after 6^{th} treatment) though there may be some cross-over into vehicle treated eye.

FIG. 4C depicts SR141716 effects do not show evidence of desensitization with repeated 7-day treatments. *, p<0.05, ***, p<0.005 by Wilcoxon signed rank test vs. 0 (0=no change).

### DETAILED DESCRIPTION

Unless defined otherwise, all technical and scientific terms used herein have the same meaning as commonly understood by one of ordinary skill in the art to which the disclosure belongs. Although any methods and materials similar to or equivalent to those described herein can be used in the practice or testing of the present disclosure, the preferred methods and materials are described below.

Disclosed are methods generally relating to glaucoma. More particularly, the present disclosure relates to methods for treating glaucoma and to methods for reducing intraocular pressure.

As used herein, “a subject in need thereof” refers to a subject having, susceptible to or at risk of a specified disease, disorder, or condition. Such subjects can be susceptible to or at elevated risk for glaucoma and intraocular pressure due to family history, age, environment, and/or lifestyle.

Based on the foregoing, because some of the method embodiments of the present disclosure are directed to specific subsets or subclasses of identified subjects (that is, the subset or subclass of subjects “in need” of assistance in addressing one or more specific conditions noted herein), not all subjects will fall within the subset or subclass of subjects as described herein for certain diseases, disorders or conditions.

As used herein, “susceptible” and “at risk” refer to having little resistance to a certain disease, disorder or condition, including being genetically predisposed, having a family history of, and/or having symptoms of the disease, disorder or condition.

In one aspect, the present disclosure is directed to a method for treating glaucoma in a subject in need thereof. The method includes: administering a cannabinoid receptor antagonist to the subject.

Suitable cannabinoid receptor antagonists include cannabinoid CB1 receptor antagonists.

Therapeutic Cannabinoid CB1 receptor antagonists include SR141716 (5-(4-Chlorophenyl)-1-(2,4-dichloro-phenyl)-4-methyl-N-(piperidin-1-yl)-1H-pyrazole-3-carboxamide), AM251 (1-(2,4-Dichlorophenyl)-5-(4-iodophenyl)-4-methyl-N-1-piperidinyl-1H-pyrazole-3-carboxamide) and AM281 (1-(2,4-Dichlorophenyl)-5-(4-iodophenyl)-4-methyl-N-4-morpholinyl-1H-pyrazole-3-carboxamide), AM4113 (5-(4-chlorophenyl)-1-(2,4-dichlorophenyl)-4-methyl-1H-pyrazole-3-carboxamide; a neutral antagonist), CP 945598 (1-[8-(2-chlorophenyl)-9-(4-chlorophenyl)-9H-purin-6-yl]-4-(ethylamino)piperidine-4-carboxamide; a high affinity competitive antagonist) and AM6538 (4-{4-[1-(2,4-dichlorophenyl)-4-methyl-3-[(piperidin-1-yl)carbamoyl]-1H-pyrazol-5-yl]phenyl}but-3-yn-1-yl nitrate; a long-term antagonist).

Suitable routes of administering therapeutic agents include topical administration.

Suitable dosages of SR141716, AM251, AM281, CP945598, AM4113 and AM6538 can range from about 1 mM to about 10 mM for topical application.

In one aspect, the present disclosure is directed to a method for reducing intraocular pressure in a subject in need thereof. The method includes: administering a cannabinoid receptor antagonist to the subject.

Suitable cannabinoid receptor antagonists include cannabinoid CB1 receptor antagonists. Particularly suitable cannabinoid CB1 receptor antagonists include SR141716, AM251, and AM6538.

Suitable subjects in need thereof include subjects having or are suspected of having glaucoma.

Suitable routes of administering cannabinoid CB1 receptor antagonists include topical administration.

Suitable dosages of SR141716, AM251, and AM6538 can range from about 1 mM to about 10 mM.

In one aspect, the present disclosure is directed to a method for treating glaucoma in a subject in need thereof, the method comprising administering a negative allosteric modulator to the subject.

Suitable negative allosteric modulators of cannabinoid CB1 receptor include PSNCBAM1 (1-(4-chlorophenyl)-3-(3-(6-(pyrrolidin-1-yl)pyridin-2-yl)phenyl)urea) and GAT100 (3-ethyl-5-isothiocyanato-N-(4-(piperidin-1-yl)phenethyl)-1H-indole-2-carboxamide).

Suitable routes of administering negative allosteric modulators of CB1 receptor include topical administration and injection.

Suitable dosages of the PSNCBAM1 and GAT100 can range from about 1 mM to about 10 mM.

In one aspect, the present disclosure is directed to a method for reducing intraocular pressure in a subject in need thereof, the method comprising administering a negative allosteric modulator to the subject.

Suitable negative allosteric modulators of cannabinoid CB1 receptor include PSNCBAM1 and GAT100.

Suitable subjects in need thereof include subjects having or suspected of having glaucoma.

Suitable routes of administering negative allosteric modulators of cannabinoid CB1 receptor include topical administration and injection.

Suitable dosages of the PSNCBAM1 and GAT100 can range from about 1 mM to about 10 mM.

### EXAMPLES

### Materials and Methods

IOP was measured in mice by rebound tonometry, using a Tonolab (Icare Finland Oy, Helsinki, Finland). This instrument uses a light plastic-tipped probe to briefly make contact with the cornea; after the probe encounters the eye the instrument measures the speed at which the probe rebounds in order to calculate IOP.

To obtain reproducible IOP measurements, mice were anesthetized with isoflurane (3% induction). The anesthetized mouse was then placed on a platform in a prone position, where anesthesia was maintained with 2% isoflurane. Baseline fop measurements are taken in both eyes. A ‘measurement’ included the average value of six readings. One eye was then treated with drug (dissolved in Tocrisolve (Tocris Biosciences), a soya-based solvent, 5 μL final volume, applied topically) while the other eye was treated with vehicle. The animal was then allowed to recover. After an hour the animal was again anesthetized as above. IOP was then measured in the drug-treated and vehicle-treated contralateral eye.

IOP measurements following drug administration were analyzed by paired t-tests comparing drug-treated eyes to contralateral vehicle-treated eyes. Where animals were injected with drug, ocular pressures of animals were compared with those of vehicle-injected animals and compared using an unpaired t-test.

### Example 1

In this Example, intraocular pressure in animals treated with SR141716 was determined.

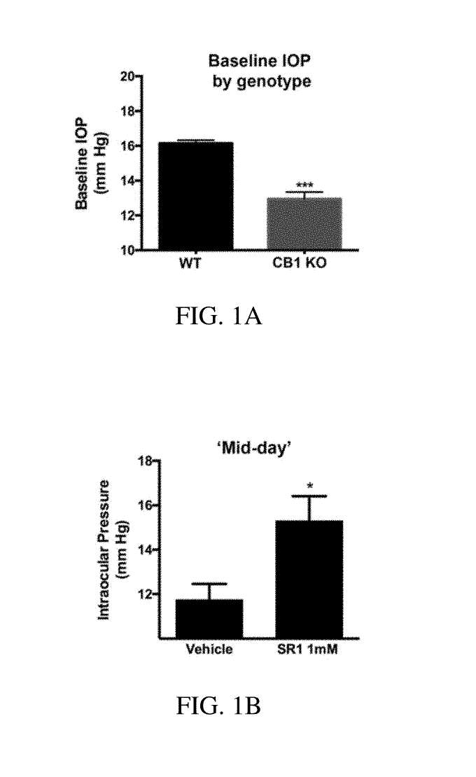

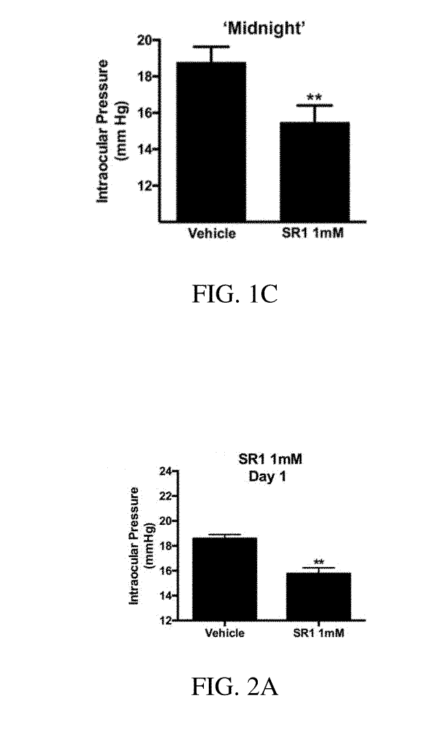

Intraocular pressure in CB1 knockout mice were compared to intraocular pressure in wild type mice. As depicted in FIG. 1A, CB1 knockout mice had lower baseline ocular pressures than wild type controls.

CB1 knockout and wild type mice were maintained in standard light cycle and in reverse light cycle. Mice were administered SR141716 (1 mM) at midday and at midnight. As depicted in FIG. 1B, SR141716 raised intraocular pressure at midday as expected in mice maintained in the standard light cycle. Surprisingly and unexpectedly, however, SR141716 reduced intraocular pressure at midnight in mice maintained in the reverse light cycle.

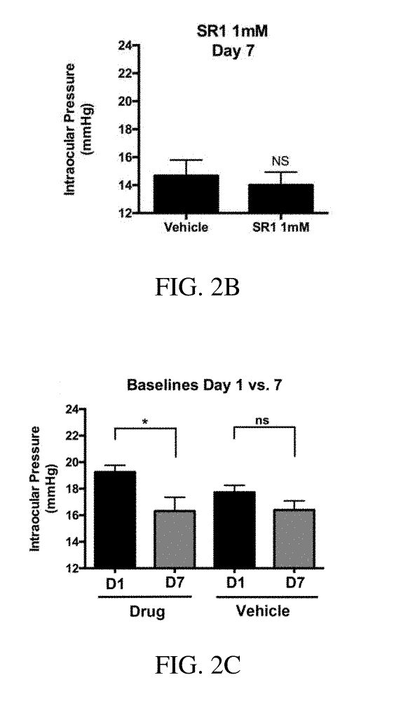

As depicted in FIG. 2C, daily treatments with SR141716 resulted in sustained lower ocular pressure. As depicted in FIG. 2A SR141716 lowered pressure on the first day of application but on the 7^{th} day, presumably because the pressures were already lowered by the previous treatment. This indicated that SR141716 effects extended beyond one day.

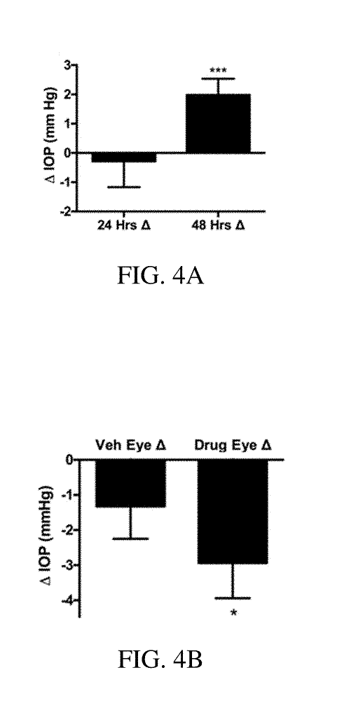

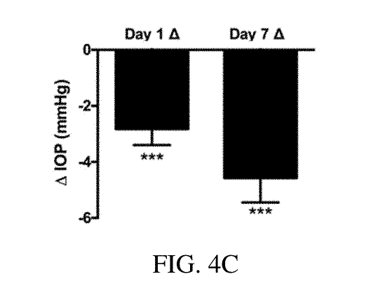

The duration was tested of the effect of SR1 with the finding that SRI effects are absent at 24 hours after a single treatment (FIG. 4A: SR141716 1 mM effect at 24 hrs (Δ IOP±SEM): −0.3±0.3; at 48 hrs: +2.0±0.6, n=16; ***, p<0.005 by Wilcoxon signed rank test vs. 0 (0=no effect)). However IOP rebounded above baseline at 48 hours.

The outcome of repeated daily treatment with SR1 was tested separately. It was found that seven days of daily treatment had several consequences. First we found that measuring IOP on the 7th day before treatment (i.e. 24 hours after the 6th daily treatment) IOP was still lowered in the drug-treated eye (FIG. 4B: SR141716 1 mM, 7 days in Vehicle-treated eye, before 7th treatment (A IOP±SEM): −1.3±0.9; drug-treated eye: −2.9±1.0, n=8; *, p<0.05 by Wilcoxon signed rank test vs. 0 (0=no effect)). The IOP was also lower in the vehicle treated eye but this difference was not statistically significant. Seven days of treatment did not result in desensitization of the IOP response (FIG. 4C: SR141716 1 mM effect at 1 day in drug-treated eye (Δ IOP±SEM): −2.8±0.6; drug-treated eye: −4.6±0.9, n=8; ***, p<0.005 by Wilcoxon signed rank test vs. 0 (0=no effect)) and ended with a drop of 4.6 mm Hg in IOP. No signs of irritation were observed after the 7 days of daily treatment.

### Example 2

In this Example, intraocular pressure in animals treated with PSNCBAM1 was determined.

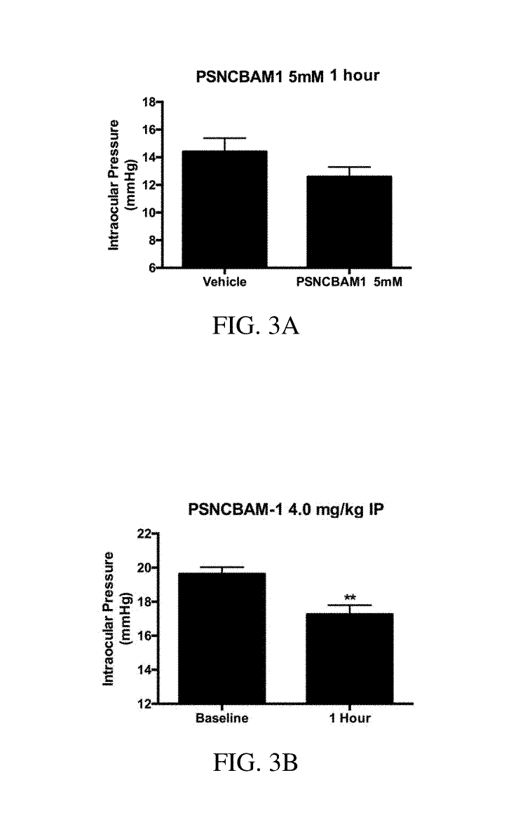

As depicted in FIG. 3A, topical treatment of WT mice with PSNCBAM1 did not lower ocular pressure relative to the contralateral eye but as shown in FIG. 3B, intraperitoneal injection lowered ocular pressure. Moreover, the apparent non-effect of topical treatment may be due to a cross-over effect since pressures in both eyes dropped after treatment despite treatment in only one eye.

More specifically, when PSNCBAM1 was administered topically, it was found to be without effect (FIG. 3A, vehicle-treated eye (mm Hg±SEM): 17.4±0.7, PSNCBAM1 (5 mM): 18.0±0.6, n=8, not significant by paired t-test). To rule out the possibility of a false-negative due to poor corneal penetration for PSNCBAM1, IOP was also tested after injection with the compound finding that this yielded a significant drop in IOP (FIG. 3B, baseline IOP (mm Hg±SEM): 19.6±0.4; 1 hour after PSNCBAM1 (4 mg/kg IP): 17.3±0.5; n=16; p<0.005 by paired t-test).

In view of the above, it will be seen that the several advantages of the disclosure are achieved and other advantageous results attained. As various changes could be made in the above methods without departing from the scope of the disclosure, it is intended that all matter contained in the above description and shown in the accompanying drawings shall be interpreted as illustrative and not in a limiting sense.

When introducing elements of the present disclosure or the various versions, embodiment(s) or aspects thereof, the articles “a”, “an”, “the” and “said” are intended to mean that there are one or more of the elements. The terms “comprising”, “including” and “having” are intended to be inclusive and mean that there may be additional elements other than the listed elements.

What is claimed is:

1. A method for treating glaucoma in a subject in need thereof, the method comprising: administering a cannabinoid receptor antagonist to the subject.

2. The method of claim 1, wherein the cannabinoid receptor antagonist is a cannabinoid CB1 receptor antagonist.

3. The method of claim 1, wherein cannabinoid CB1 receptor antagonist is selected from SR141716, AM251, AM281, CP945598, AM4113 and AM6538.

4. The method of claim 1, wherein the cannabinoid receptor antagonist is administered at a dosage ranging from about 1 mM to about 10 mM.

5. A method for method for reducing intraocular pressure in a subject in need thereof, the method comprising: administering a cannabinoid receptor antagonist to the subject.

6. The method of claim 5, wherein the cannabinoid receptor antagonist is a cannabinoid CB1 receptor antagonist.

7. The method of claim 6, wherein cannabinoid CB1 receptor antagonist is chosen from SR141716, AM251, and AM6538.

8. The method of claim 5, wherein the subject has or is suspected of having glaucoma.

9. The method of claim 5, wherein the cannabinoid receptor antagonist is administered at a dosage ranging from about 1 mM to about 10 mM.

10. A method for treating glaucoma in a subject in need thereof, the method comprising: administering a negative allosteric modulator of a cannabinoid receptor to the subject.

11. The method of claim 10, wherein negative allosteric modulator of a cannabinoid receptor is a negative allosteric modulator of a cannabinoid CB1 receptor.

12. The method of claim 11, wherein the negative allosteric modulator of a cannabinoid CB1 receptor is chosen from PSNCBAM1 and GAT100.

13. The method of claim 10, wherein the cannabinoid receptor antagonist is administered at a dosage ranging from about 1 mM to about 10 mM.

14. A method for method for reducing intraocular pressure in a subject in need thereof, the method comprising: administering a negative allosteric modulator of a cannabinoid receptor to the subject to the subject.

15. The method of claim 14, wherein negative allosteric modulator of a cannabinoid receptor is a negative allosteric modulator of a cannabinoid CB1 receptor.

16. The method of claim 15, wherein the negative allosteric modulator of a cannabinoid CB1 receptor is chosen from PSNCBAM1 and GAT100.

17. The method of claim 14, wherein the subject has or is suspected of having glaucoma.

18. The method of claim 14, wherein the cannabinoid receptor antagonist is administered at a dosage ranging from about 1 mM to about 10 mM.
